(19) United States
(12) Patent Application Publication
(10) Pub. No.: US 2025/0329436 A1
(43) Pub. Date: Oct. 23, 2025

(54) CALCULATION METHOD FOR NUCLEAR MEDICINE BRAIN FUNCTIONAL IMAGING TEMPLATE

(71) Applicant: National Atomic Research Institute, Taoyuan (TW)

(72) Inventors: FAN-PIN TSENG, Taoyuan (TW); Yu-ching Ni, Taoyuan (TW)

(21) Appl. No.: 19/047,881

(22) Filed: Feb. 7, 2025

(30) Foreign Application Priority Data
Apr. 23, 2024 (TW) 113115147

(51) Int. Cl. G16H 30/40; G16H 10/60; G16H 50/20; G16H 50/70

(52) U.S. Cl. CPC G16H 30/40; G16H 10/60; G16H 50/20; G16H 50/70

(57) ABSTRACT

A calculation method for a nuclear medicine brain functional imaging template includes the following steps: selecting multiple sets of images from a known healthy human database; defining a position-age function by a position information in the set of images and an age information corresponding to the image; utilizing machine learning to compute the position-age function for obtaining a machine learning model and obtaining a weight information correspondingly; and calculating an expected value template function corresponding to the machine learning model based on the weight information and the age information.

### CROSS REFERENCE TO RELATED APPLICATION

This application claims the benefits of Taiwan application Serial No. 113115147, filed on Apr. 23, 2024, the disclosures of which are incorporated by references herein in its entirety.

### TECHNICAL FIELD

The disclosure relates to a calculation method, in particular to a calculation method for a nuclear medicine brain functional imaging template.

### BACKGROUND

With the aging of society, the number of dementia patients will increase significantly, and the consequent demand and cost of care services are high compared to other people with disabilities. Therefore, early detection of dementia and treatment to delay the deterioration of the disease have become an important issue for the society nowadays. Currently, the diagnosis of dementia patients requires necessary examination procedures to identify possible types of dementia from a variety of evidence.

The conventional approach is to calculate the expected value and standard deviation of a group of healthy human brain images for an age range in the nuclear medicine brain functional imaging database for each voxel as a statistical template. The method of use is subtracting the input image from the expected value and dividing it by the standard deviation (statistically known as the Z-score), which will highlight the “differences between healthy brain images” and provide doctors with an auxiliary reference for identifying dementia.

However, the traditional statistical data for age ranges are cross-sectional and non-continuous. If the segmenting method of 61˜70 years old as an age range and 71˜80 years old as an age range, the brain area standard template for 61 years old and the brain area standard template for 69 years old have the same expectation value for healthy people. The same brain standard template will be adopted in the same interval. As a result, it leads to the lack of ability to differentiate between these ages in the same range of 61˜70 years old. Additionally, there is no continuity in changes across different intervals. For example, the standard templates of brain areas at 70 and 71 years old are very different. If the age range is segmented very finely, a certain level of data must be collected for each age group to make a standard brain area template. However, it is difficult to obtain nuclear medicine images, and there are even fewer images of healthy people. This means that it is difficult to obtain nuclear medicine imaging data in reality, and enough nuclear medicine brain functional imaging data cannot be obtained, Consequently, the amount of imaging data is too limited to subdivide the age range.

Furthermore, the brain area is a group of voxel sets. However, the Z-score calculation method of the traditional method merely considers a single voxel. It only quantifies areas such as the cerebellum or the whole brain, and then divides the entire image by the quantitative value to correct the absorption activity, without considering the relationship between adjacent voxels and the overall changes in the brain area, which is easily interfered by noise in the image. For example, some voxels in the image brain area of healthy people are caused by broken instruments or other imaging errors (such as imaging software calculation or alignment errors), thereby increasing the expected value and standard deviation.

In addition, the imaging procedures of different hospitals or the imaging methods of different brands of instruments vary slightly, and there is a lack of image judgment standards across hospitals and different brands of instruments. Therefore, it is difficult to reflect individual differences by using a uniform brain standard template, and it is not easy to make corrections across different hospitals and different brands.

Moreover, the definition of health in the images for healthy people tends to be qualitative, and there may be sub-healthy images mixed in. For example, the collection process of images of healthy people is based on patients with suspected dementia. After nuclear medical imaging studies, it is determined that they “should” be normal and are considered normal. It has been collected into the nuclear medicine brain functional imaging database, but there are a small number of abnormalities in the brain area, resulting in errors in the Z-score image calculation.

### SUMMARY

A calculation method for nuclear medicine functional brain imaging templates is provided by the embodiment of the disclosure. It can provide and generate the expected value template function and standard deviation template function of healthy people under limited data, and provide a computer-assisted judgement on abnormal characteristics of nuclear medicine brain functional images.

The embodiment of the disclosure provides a calculation method for a nuclear medicine brain functional imaging template, which can be read by a computer for execution. The computer includes a controller. The computer is suitable for connecting to an imaging instrument. The calculation method for a nuclear medicine brain functional imaging template includes the following steps: selecting multiple sets of images from a known healthy human database; defining a position-age function by associating a position information in the set of images with an age information corresponding to the image; utilizing machine learning to compute the position-age function for obtaining a machine learning model and obtaining a weight information correspondingly; and calculating an expected value template function corresponding to the machine learning model based on the weight information and the age information.

A detailed description is given in the following embodiments with reference to the accompanying drawings, in order to make the disclosure more comprehensible.

### BRIEF DESCRIPTION OF THE DRAWINGS

FIG. 1 is a schematic diagram of a nuclear medicine brain functional imaging template calculation system according to the disclosure.

FIG. 2 is a flow chart of the calculation method of the nuclear medicine brain functional imaging template according to the disclosure.

FIG. 3 is a schematic diagram of an embodiment of the age and the position-age function according to the disclosure.

FIG. 4 is a schematic diagram of an embodiment of the age and standard deviation function according to the disclosure.

### DETAILED DESCRIPTION

The following embodiments are set forth in detail with accompanying drawings, but the embodiments provided are not intended to limit the scope of the disclosure. In addition, the drawings are for illustrative purposes only and are not drawn to original size. To facilitate understanding, the same components will be identified with the same symbols in the following description.

The terms “including”, “comprising”, “having”, etc. mentioned in the disclosure are open terms, which means “including but not limited to”.

In the description of various embodiments, when describing the components in terms of “first,” “second,” “third,” “fourth,” and the like, it is used only to distinguish these components from one another, and does not limit the order or importance of these components.

In the illustrations of various embodiments, the so-called “coupling” or “connection” may refer to two or more elements being in direct physical or electrical contact with each other, or in indirect physical or electrical contact with each other, and “coupling” or “connection” may also refer to the mutual operation or action of two or more components.

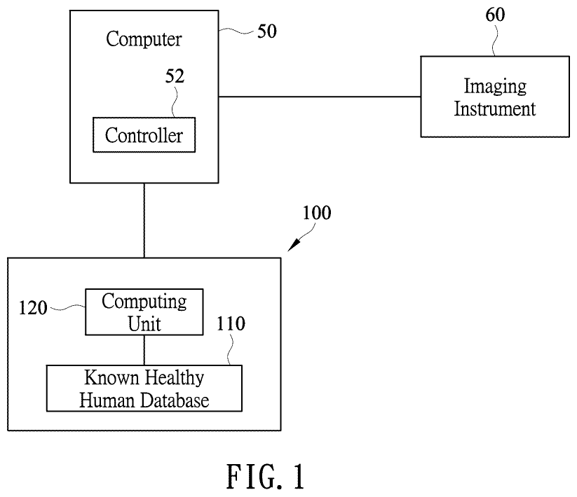

FIG. 1 is a schematic diagram of a nuclear medicine brain functional imaging template calculation system according to the disclosure. Please refer to FIG. 1. The nuclear medicine brain functional imaging template calculation system 100 of the disclosure includes a known healthy human database 110 and a computing unit 120. The computing unit 120 is connected to the known healthy human database 110 via signals. The computing unit 120 can be a software program.

The computer 50 is connected to the nuclear medicine brain functional imaging template calculation system 100 and the imaging instrument 60 via signals, so as to realize the data processing and action control of the entire nuclear medicine brain functional imaging template calculation system 100. The controller 52 can be a physical circuit to perform action control and signal transmission on the internal drives or actuators of various imaging instruments 60. The computer 50 executes the software programs which are internally stored. The controller 52 can be installed within the computer 50, or the controller 52 can be built in the software program of the computing unit 120. In addition, the computing unit 120 (such as the software program) is stored in the storage drive within the computer 50 and is read by the computer 50 to perform a series of expected method steps.

As stated in the disclosure, multiple sets of images, which may be referred to as nuclear medicine images, are obtained from brain imaging by the imaging instrument 60 and pre-processed by the computing unit 120 using the following techniques to store the images in the known healthy human database 110. The pre-processing includes the following steps: a standardized alignment procedure for the sets of images, and a numerical normalization for the sets of images.

Considering that these images are generated from the imaging instruments 60 of various hospitals, these imaging instruments 60 have different settings according to different hospitals or needs, or these imaging instruments 60 are different apparatuses. Therefore, a normalized alignment procedure is required to align the features of the sets of images to a standard position or reference frame.

The normalized alignment procedure includes extracting key feature points (such as edge points, or significant local features) from the image, describing the extracted feature points such as using the feature vectors to describe the local image information around each feature point, matching the feature description to be matched with the feature description in the aforementioned reference frame to find similar feature points, and performing alignment or positioning based on the matching feature points to align the feature points in the image into the reference frame. Furthermore, the accuracy of the alignment results can be increased and ensured by optimizing and verifying the alignment results, and the subsequent processing of the sets of images can be facilitated by normalizing the alignment procedures.

In one embodiment, the normalized alignment procedure may be performed by SPM (Statistical Parametric Mapping) software, or may be calibrated through prosthesis experiments.

In addition, normalization is the process of scaling the image information of the original data to the range of 0˜1 without changing the original distribution, so as to eliminate the influence of different units and develop comparability between different variables. For example, all voxels of the whole image are divided by the expected value of the non-zero voxels in the cerebellar region, and the convolution is performed for the images with an appropriate Blur function to reduce the background noise or the alignment error.

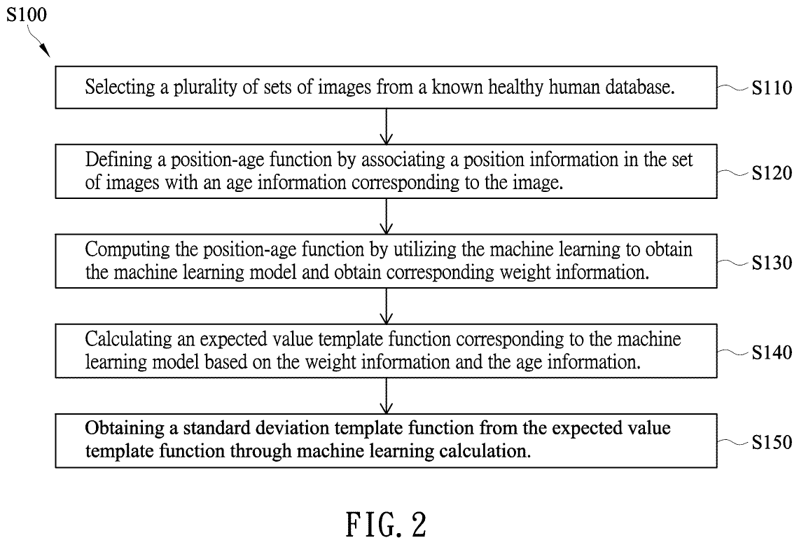

FIG. 2 is a flow chart of the calculation method of the nuclear medicine brain functional imaging template according to the disclosure. Please refer to FIGS. 1 to 2. The disclosed calculation method S100 for the nuclear medical brain functional imaging template includes the following steps S110 to S150. The calculation method S100 for the nuclear medical brain functional imaging template is, for example, suitable to be established in the computing unit 120 (such as a software program or a firmware program), stored in the storage drive of a computer 50 or controller 52 and executed by the computer 50.

First, step S110 is performed to select a plurality of sets of images from a known healthy human database 110, wherein these sets of images are images of healthy people. Of course, these sets of images can be pre-processed according to actual conditions, such as normalized alignment procedures and numerical regularization of these sets of images.

After the above step S110, step S120 is executed to define a position-age function f(x,t) by associating a position information x in the set of images with an age information t corresponding to the image to establish the expected value template function fₘₑₐₙ(x, t) and standard deviation template function f_{std}(x, t) for a brain area.

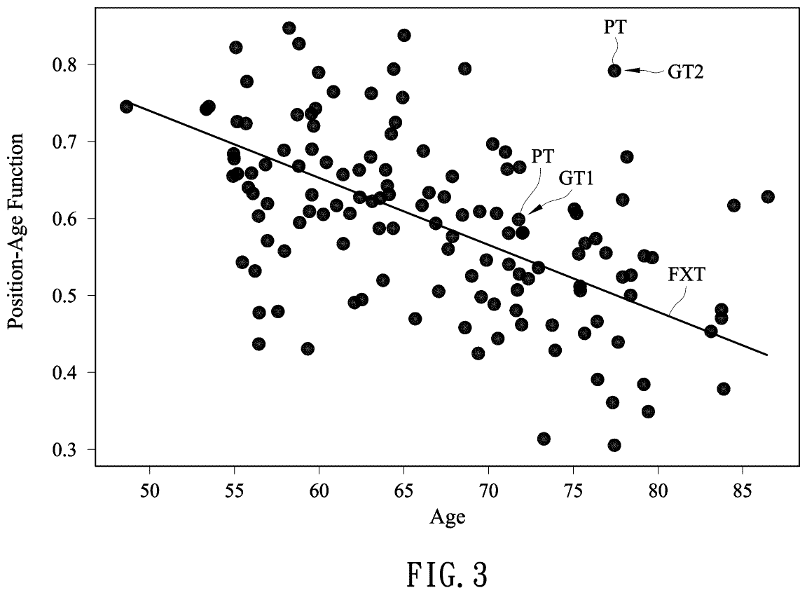

The position-age function f(x,t) of the disclosure represents the functional relationship between the position information x and the age information t of each position. For example, for the position information x of a specific or fixed position, multiple position-age functions f(x,t) are selected as illustrated in FIG. 3. The horizontal axis of FIG. 3 is age. For example, take the age information x of age 50˜85, and the vertical axis of FIG. 3 is the position-age function f(x,t). For example, the position-age function f(x,t) is 0.3˜0.9 corresponding to the aforementioned age information x of age 50˜85, and each data point PT represents the value of a certain age and the corresponding position-age function f(x,t).

After the above step S120, step S130 is executed to compute the position-age function f(x,t) by utilizing the machine learning to obtain the machine learning model and obtain corresponding weight information. Among them, machine learning models are mathematical or logical models established by machine learning algorithms. Machine learning models include linear regression models, decision tree models, support vector machine models (SVM Models), artificial neural network models (ANN Models), clustering models, generative models, etc. The machine learning models can be adjusted appropriately according to the actual situation.

For example, a linear regression model is used for computing. In association with FIG. 3, a linear function is used in a machine learning manner to obtain and establish the relationship between the independent variable and the dependent variable. Among them, the independent variable is the position-age function f(x,t), and the dependent variable is the age information t. In other words, a machine learning model in which the position-age function f(x,t) varies with the age information t is obtained through a linear regression method.

The above machine learning model can be described by the following mathematical formula (1):

$$f ( x , t ) = y = w \times t + b \qquad ( 1 )$$

In the above mathematical formula (1), f(x, t) is the position-age function, t is the age information. The position-age function f(x, t) changes with the age information t, and is represented by the regression line FXT of the linear function. Additionally, (w,b) is the weight information of the machine learning model.

Assuming that the image size is 256×256, there will be 256×256 sets of weight information (w,b) to describe the changes in each voxel of each healthy person's image at different ages. If the number of images of healthy people is insufficient, there will be error in the calculation of the weight information (w,b) of the machine learning model, resulting in the error of the above data points PT and the error of the regression line FXT of the linear regression method to be larger. For example, the first data GT1 of PT in these data points is closer to the regression line FXT, but the second data GT2 is farther away from the regression line FXT.

In one embodiment, a sufficient number of images of healthy people can be used to supplement the machine learning model to reduce the margin of error obtained by the above machine learning algorithm for the position-age function f(x,t).

In the embodiment, how to correct the weight information in case of a limited or insufficient number of images of healthy human in the actual situation. In the above step S110, multiple sets of images are selected from the known healthy human database 110. These sets of images are randomly selected batch data for training the machine learning model in the subsequent step S130.

Step S130 includes the following steps: training the machine learning model with a batch of the position-age functions f(x,t) and the age information t, having a Loss function, and using a gradient descent method (Gradient Descent) to correct the weight information (w,b). Batch processing can provide more stable data estimation, reduce storage requirements, and can be used for simultaneous computing (parallel computing), so that multiple batches of batch data can be calculated in parallel to improve training efficiency.

For example, during each weight information (w,b) calculation of the linear regression model with f(x, t)=y=w×t+b, a batch (e.g., 10) of the position-age function f(x,t) and age information t are selected from the known healthy human database 110, and the position-age function f(x,t) and age information t of these 10 positions are calculated based on w×t+b to obtain y′. Afterwards, y′ from the actual y (that is, f(x,t)) is calculated as the distance between the two. For example, with ∥y′−y∥, the distance between the two can be regarded as the above loss function. For example, the loss function calculated by the second data GT2 will be relatively greater than the loss function calculated by the first data GT1. Consequently, in the process of training the machine learning model, the weight information (w,b) is trained by the position-age function f(x,t) and the age information t of the batches, and compared with the actual y (i.e., f(x,t)). The weight information (w,b) can be corrected and the loss function can be reduced by the continuous training process.

In one embodiment, for example, the gradient descent method can be used to correct the weight information (w,b). The gradient descent method is one of the most commonly used optimization algorithms in machine learning and is used to solve the minimum value of the objective function (such as the loss function in the embodiment). The main steps are calculating the partial derivative (gradient) of the loss function and each weight information (w,b), then updating the weight information (w,b) in the opposite direction of the gradient to reduce the value of the loss function, and repeat the iteration to correct the weight information (w,b) so that the loss function can reach the minimum value.

If the data of the sets of images stored in the known healthy human database 110 is good, the loss function obtained in the above steps will be relatively low. Accordingly, the training process will be accelerated and the calculated machine learning model will be more focused and accurate. However, if the data of the sets of images stored in the known healthy human database 110 is insufficient or not good enough (for example, the data difference is too large, which may be caused by the different sample quality in each hospital), this will lead to the loss function obtained from the above steps relatively high. Of course, it is also possible to gradually reduce the loss function of the data through the training process, in order to calculate the machine learning model. Therefore, in one embodiment, step S110 can further include the following step: setting a threshold to exclude outliers from the set of images. Since the sets of images in the known healthy human database 110 may be obtained from different hospitals, the same hospital, or different imaging instruments 60, the qualities of the obtained images are different. In order to improve the accuracy of the standard templates (e.g., the expected value template function fₘₑₐₙ(x, t)) that are subsequently fitted to the brain areas of different ages by machine learning, a threshold is set to exclude or select elements within the appropriate range.

In other words, by setting a threshold, images with special conditions are initially excluded (if the data of images with special conditions are significantly different from most data, they can be called outliers). The set of images within the threshold can be gathered together as a batch for performing the machine learning algorithms to obtain the machine learning model, so as to speed up the computing speed and obtain the machine learning model more accurately.

It should be noted that the numerical definition of the above threshold is based on the actual situation of the machine learning model to be calculated, or based on the preliminary observation of the numerical values of these sets of images stored in the known healthy human database 110. If FIG. 3 is taken as an example, the first data GT1 of PT in these data is closer to the regression line FXT but the second data GT2 is farther away from the regression line FXT, the second data GT2 can be set as an outlier.

During the above steps of eliminating outliers in these sets of images, an example to illustrate how to define outliers can include the following steps: for the age information t in these sets of images, calculating the loss function of the age information t, and integrating these loss functions into a loss set function. For example, the difference between y′ calculated for all age information t and the actual y (i.e., f(x,t)) is used to obtain a loss set function S of all the differences, and the loss set function S is used to obtain a loss set function S of all the differences. If the difference of the loss set function S is too large, such as exceeding the above threshold, or is a single outlier different from the data range of the most of the differences (e.g., if the data range of most of the differences is from 10 to 20 but the difference is 0.002, it is obviously different from the data range of the above differences). Taking FIG. 3 as an example, if the difference of the second data GT2 in the loss set function S is too large, the second data GT2 can be set as an outlier.

After the step of integrating the loss functions of these age information into a loss set function, afterwards, the corresponding loss function whose difference in the loss set function is greater than the threshold is defined as the outlier. For example, a confidence interval of a certain proportion (such as 95%) is regarded as a threshold, and the difference in the loss set function exceeds the confidence interval of this proportion (i.e., the threshold), is set to exclude or reduce the selection of images with this difference (such as the second data GT2 in FIG. 3), so as to ensure that the difference between the selected sets of images are within the threshold, or that the set of images with large differences in the loss function can be reduced into subsequent machine learning calculations, thereby decreasing the additional calculation steps performed subsequently to reduce the difference in loss functions.

In addition to the above embodiment of defining outliers, in other embodiments, the step of excluding the outliers of the sets of images includes the following steps: data are classified into a number of tiers based on the extent of image anomalies in the known healthy human database 110. For example, the images can be blindly tested by a doctor to evaluate the healthy or normalcy degree in order to establish a classification data of severity grading in the known healthy human database 110. Blind testing refers to the process of conducting a test or assessment without knowledge or information in advance (e.g. whether the image is good or bad, or from which hospital the image was obtained) for eliminating the influence of subjective bias or preconceived ideas, in order to obtain a more objective and accurate result.

After the classification is completed, a corresponding sampling rate value is set based on the classification data. If the sampling rate value is less than the threshold, the outlier is defined. For example, in the process of obtaining these position-age functions f(x,t) and age information t in batches from the known healthy human database 110, uneven sampling is used based on the classification data of the severity of the image. The uneven sampling means selecting the images with unequal probability. For example, the classification data is divided into levels 0 to 4 according to the severity of the image, wherein level 0 indicates the healthiest, level 3 indicates that the image is suspected to be abnormal. The sampling rate value is set to level 0 for 0.4, level 1 for 0.3, level 3 for 0.2, and level 4 for 0.1. That is, the higher sampling rate value is set to level 0, and the lowest one is level 4. The threshold can be set to 0.2, for example. If the sampling rate value is less than the threshold, it is defined as an outlier, to avoid or reduce the classification data of serious or unqualified images below the threshold from entering the subsequent calculation process. For example, taking FIG. 3 as an example, the first data GT1 of these data PT is rated as level 0, but the second data GT2 may be rated as level 4.

Especially when the images of interest (such as the image with healthiest level 0) are unevenly distributed in all images, the uneven sampling method can be used for more targeted selection of images of interest (e.g., setting the image with healthiest level 0 to have a high probability of sampling value) to ensure that a batch of these position-age functions f(x,t) and the age information t in the process have better representative of the characteristics of the overall population. This can prevent similar sub-healthy images from being mixed in. For example, the image collection process of healthy people is based on a patient suspected of dementia. After nuclear medical imaging, it should be determined normal. However, in fact, the brain area of the patient has some abnormalities. If the sub-healthy image is added into the calculation, the calculation process will be biased.

In addition to the above sampling rate value to avoid selecting sub-healthy images, in other embodiments, after the classification is completed, the classification data are then multiplied by a fixed value to obtain multiple sampling classification data. For example, each of the above four levels 0 to 4 is multiplied by a fixed value of 0.3 to obtain four sampling classification data of 0, 0.3, 0.6, and 0.9 respectively. Afterwards, the probability distribution of a Gaussian distribution curve with a fixed σ (sigma) is taken to be random, and then the absolute value is obtained to r as the threshold. For example, the probability of being less than one time of σ (sigma) is 34.1%+34.1%, which is 68.2. Compare the above sampling classification data with the threshold r. If the sampling classification data is greater than the threshold, it is defined as an outlier. For example, when the sampling classification data is 0, it means that a threshold greater than 0 needs to be selected, which means that the image will be selected definitely. On the other hand, when the sampling classification data is 0.9, it means that the image will be selected only if the threshold is greater than 0.9. If σ(sigma) is 0.9, it equals to about 31.8% of the probability of selecting the image.

However, the above selection using the probability of Gaussian distribution is an implementation embodiment. In other embodiments, other statistical probability distribution settings can be used to make it easier for the images of interest to be selected.

In one embodiment, compared with the conventional Z-score calculation method, it only considers a single voxel, quantifies only the area such as cerebellum or the whole brain, and then divides the whole image by the quantitative value to correct the absorptive activity. It does not consider the relationship between the neighboring voxels and the overall changes in the brain area, and is susceptible to the interference of noise in the image. Therefore, in the disclosure, for different position information x in each set of images, it is necessary to consider that the weight information of adjacent voxels of each position information x needs to be close, or the weight information of the same brain area needs to be close. The above step S130 may include the following steps: based on the characteristics of brain area, distinguishing multiple brain-area locations, such as the cerebrum, cerebellum, or left brain, right brain, or further distinguishing the four brain lobes of frontal lobe, parietal lobe, temporal lobe and occipital lobe. The brain area to be interested can be determined according to the actual situation.

Afterwards, the weight information corresponding to the position information in the same brain area is compared to obtain a position-loss function. For example, a position value in the position information is calculated to obtain the weight information of the position value. Afterwards, multiple neighboring position values with adjacent position values are calculated to obtain the weight information corresponding to these neighboring position values, and then the weight information of the neighboring position values are compared with the weight information corresponding to the position values to obtain a position-loss function.

Take the neighboring voxels of a certain position information as an example, a range is set. For example, there are 26 voxels near the top, bottom, left, right, front and back of a certain position information, these 26 voxels have their corresponding weight information, and have their corresponding loss functions. The loss functions are compared to find an appropriate position-loss function for these loss functions. It should be noted that the position-loss function does not use these loss functions as an average, but sets an appropriate mask based on the voxel proximity and brain-area characteristics in a brain area, and calculates the difference with the weight information, to set the appropriate positional loss function in that same brain area.

In one embodiment, a gradient descent method is further utilized to calculate the position-loss function, so that during the gradient descent process, the relationship with the adjacent voxels is synchronously approximated to correct the weight information. In other embodiments, during the above computing process of calculating the position-loss function, regularization technology, such as Lasso or Ridge algorithm, can be added based on situations to make the result of calculating the position-loss function more stable, as shown in the following mathematical formula (2) which describes:

$$‖ {y}^{′} - y ‖ + α ‖ W ‖ + β {\sum }_{i , j ∈ mask} ‖ {w}_{i} - {w}_{j} ‖ \qquad ( 2 )$$

Among them, ∥y∧′−y∥ is the loss function design corresponding to the linear regression model technology in step S130 mentioned above, and it can be calculated using MSE (Mean Square Error) or MAE (Mean Absolute Error). ∥W∥ corresponds to the regularization technique, and ∥wi−wj∥ is the correction amount corresponding to the calculation of the loss function when voxels i and j belong to the same mask (such as adjacent voxel groups or the same brain area), α(alpha) and β(beta) are algorithm hyperparameters used to control the ration between ∥y′−y∥, ∥W∥, and ∥wi−wj∥. For example, different β (beta) can be set for different brain area sizes, to reduce or enhance the consistency of changes in the linear regression of different voxels in the brain area.

After step S130, step S140 is executed to calculate an expected value template function fₘₑₐₙ(x, t) corresponding to the machine learning model based on the weight information and the age information. The weight information can be modified by the loss function in the previous step. For example, the above method can be used to generate the position-age function f(x,t) of all voxels in the set of images, and then substitute different age information t to further obtain the expected value template function fₘₑₐₙ(x, t) of the age information t.

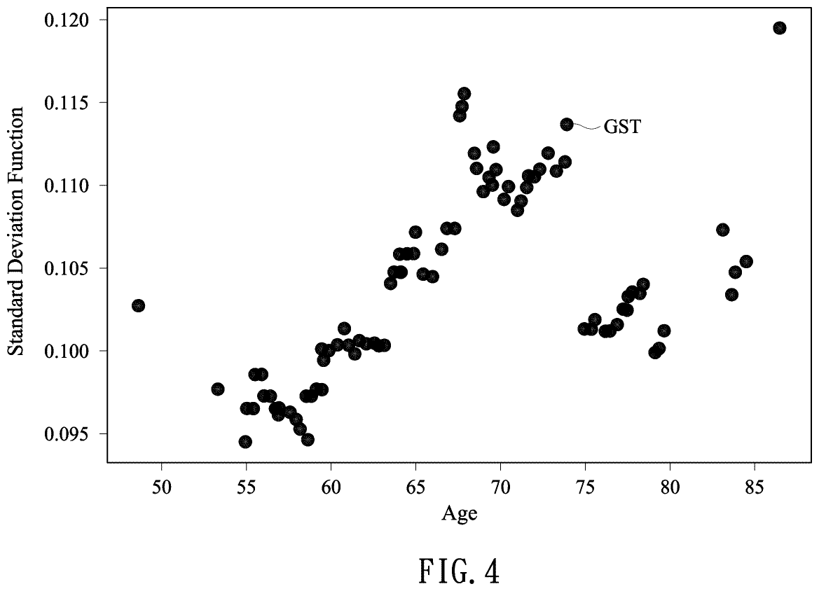

After the expected value template function fₘₑₐₙ(x, t) of the age information t is obtained in the above step S140, step S150 is then performed to obtain a standard deviation template function f_{std}(x, t) from the expected value template function fₘₑₐₙ(x, t) through machine learning calculation, which includes the following steps: First, a standard deviation function is calculated for an age range interval in the expected value template function f_{std}(x, t). For example, for a fixed position information x, an age range interval is set and further statistics is performed to obtain the standard deviation. The range is assumed to be 10 for calculating FIG. 3. For the case of age information t=60, take all the data of age information t=55 to 65 (centered on 60, range 10 for sampling), calculate the distance to the expected value template function fₘₑₐₙ(x, t), add the squares and divide by the total number, and take the root sign to get the result of age information t=60. Afterwards, the age information t keeps changing, such as age information t=61, age information t=62, they both use range 10 for sampling. After repeating the above method to process all the age information t, the schematic diagram of the relationship between the age information and the standard deviation function in FIG. 4 is obtained. Of course, after it is done for a fixed position information x, it turns to the next fixed position information x and repeat the above for all age information t. As shown in FIG. 4, all the data of age information are listed, and the horizontal axis of FIG. 4 is age. For example, the age information x of age 50˜85 is taken. The vertical axis of FIG. 3 is the positional age function f(x,t). For example, the standard deviation function of 0.095˜0.120 can be obtained corresponding to the age information x of age 50˜85. Each data point GST represents the value of the standard deviation function between a certain age and the corresponding standard deviation function, and thus the above calculation is used to obtain the result of age information t=48 to t=86.

Next, the machine learning is used to calculate the standard deviation function to obtain a standard deviation machine learning model, and the corresponding standard deviation weight information is obtained. The calculation method for the step is similar to step S130. They merely differ in replacing the position-age function f(x, t) with the standard deviation function, to obtain the standard deviation machine learning model and the corresponding standard deviation weight information. The detailed steps performed in the above step S130 can also be applied. Afterwards, the standard deviation template function f_{std}(x, t) corresponding to the standard deviation machine learning model is calculated based on the standard deviation weight information and age information.

In summary, the calculation method of the nuclear medicine brain functional imaging template disclosed in the disclosure is an algorithm for generating statistical templates for the nuclear medicine brain functional imaging database. The machine learning technology is used by the algorithm to fit the standard templates of brain areas of different ages (i.e., the expected value template function and the standard deviation template function). The machine learning techniques utilize algorithms such as spatial convolution, regularization, and ensemble learning, and it consider factors such as “spatial neighboring voxels,” “severity of healthy people,” “overall characteristics of the brain area,” and “image outliers” to establish coefficient weights that vary according to age, so that when the healthy human database is limited, the brain area standard template can be established for various application scenarios.

Although the disclosure has been disclosed in the form of embodiments, it is not intended to limit the present disclosure. Anyone with general knowledge in the field of technology may make some changes and embellishments without departing from the spirit and scope of the present disclosure, and therefore the scope of protection of the disclosure shall be subject to the scope of the patent application attached hereto.

What is claimed is:

1. A calculation method for a nuclear medicine brain functional imaging template, suitable for being established in a software program and read by a computer to perform the following steps:
selecting multiple sets of images from a known healthy human database;
defining a position-age function by associating a position information in the set of images with an age information corresponding to the image;
utilizing machine learning to compute the position-age function for obtaining a machine learning model and obtaining a corresponding weight information; and
calculating an expected value template function corresponding to the machine learning model based on the weight information and the age information.

2. The calculation method for a nuclear medicine brain functional imaging template according to claim 1, wherein the step of selecting multiple sets of images from the known healthy human database comprises the following step:
setting a threshold to exclude an outlier from the sets of images.

3. The calculation method for a nuclear medicine brain functional imaging template according to claim 2, wherein the step of excluding the outlier from the sets of images comprises the following steps:
for the age information in the sets of images, calculating a loss function of the age information and integrating the loss function of the age information into a loss set function; and
defining the loss function corresponding to a difference in the loss set function that is greater than the threshold as the outlier.

4. The calculation method for a nuclear medicine brain functional imaging template according to claim 2, wherein the step of excluding the outlier from the sets of images comprises the following steps:
dividing into multiple classification data based on degree of image severity in the known healthy human database;
setting a corresponding sampling rate value based on the classification data; and
defining the sampling rate value which is less than the threshold as the outlier.

5. The calculation method for a nuclear medicine brain functional imaging template according to claim 2, wherein the step of excluding the outlier from the sets of images comprises the following steps:
dividing into multiple classification data based on degree of image severity in the known healthy human database;
multiplying the classification data by a fixed value to obtain multiple sampling classification data; and
defining the sampling classification data which is greater than the threshold as the outlier.

6. The calculation method for a nuclear medicine brain functional imaging template according to claim 1, wherein the step of utilizing the machine learning to compute the position-age function for obtaining the machine learning model and obtaining the corresponding weight information comprises the following steps:
calculating a position value in the position information to obtain the weight information corresponding to the position value;
calculating multiple neighboring position values adjacent to the position value to obtain the weight information corresponding to the neighboring position values;
comparing the weight information corresponding to the neighboring position values with the weight information corresponding to the position value to obtain a position-loss function; and
computing the position-loss function by using a gradient descent method to correct the weight information.

7. The calculation method for a nuclear medicine brain functional imaging template according to claim 1, wherein the step of utilizing the machine learning to compute the position-age function for obtaining the machine learning model and obtaining the corresponding weight information comprises the following steps:
distinguishing multiple brain-area locations based on brain-area characteristics;
comparing the weight information corresponding to the position information in the same brain-area location to obtain a position-loss function; and
computing the position-loss function by using a gradient descent method to correct the weight information.

8. The calculation method for a nuclear medicine brain functional imaging template according to claim 1, wherein the step of utilizing the machine learning to compute the position-age function for obtaining the machine learning model and obtaining the corresponding weight information comprises the following steps:
training the machine learning model by using the position-age functions and the age information of a batch; and
using a gradient descent method to correct the weight information.

9. The calculation method for a nuclear medicine brain functional imaging template according to claim 1, wherein the step of utilizing the machine learning to compute the position-age function for obtaining the machine learning model comprises the following step:
using a linear regression model for computing.

10. The calculation method for a nuclear medicine brain functional imaging template according to claim 1, wherein the step of utilizing the machine learning to compute the position-age function for obtaining the machine learning model comprises the following step:
using an artificial neural network model for computing.

11. The calculation method for a nuclear medicine brain functional imaging template according to claim 1, wherein the step of calculating the expected value template function corresponding to the machine learning model based on the weight information and the age information comprises the following step:
obtaining a standard deviation template function from the expected value template function through machine learning computation comprises the following steps:
calculating a standard deviation function for an age range interval in the expected value template function;
using machine learning to compute the standard deviation function to obtain a standard deviation machine learning model and obtain a corresponding standard deviation weight information; and
calculating the standard deviation template function corresponding to the standard deviation machine learning model based on the standard deviation weight information and the age information.
